(19) United States
(12) Patent Application Publication
(10) Pub. No.: US 2024/0082484 A1
(43) Pub. Date: Mar. 14, 2024

(54) DAILY TESTOSTERONE DOSING USING WEARABLE DEVICE

(71) Applicant: Jeffrey Nuziard, Colleyville, TX (US)

(72) Inventor: Jeffrey Nuziard, Colleyville, TX (US)

(21) Appl. No.: 18/243,977

(22) Filed: Sep. 8, 2023

Related U.S. Application Data
(60) Provisional application No. 63/404,747, filed on Sep. 8, 2022.

(51) Int. Cl. A61M 5/142; A61K 31/568; A61M 5/158

(52) U.S. Cl. CPC A61M 5/14248; A61K 31/568; A61M 5/158

(57) ABSTRACT

A wearable testosterone drug delivery system including a wearable pump configured to be worn by a user overnight. During the night the pump injects a prescribed per patient dosage of a liquid form bio-identical testosterone over a predetermined time period, such as a 6-hour period, mimicking the body's natural way of hormone development. This injection mimics the human process. The pump includes a cartridge pre-loaded with TRT from a pharmacy. Each cartridge has a 31-gauge needle approximately 3/16″-¼″ in length.

### CLAIM OF PRIORITY

This application claims priority of U.S. Patent Ser. No. 63/404,747 filed Sep. 8, 2022, entitled “Daily Testosterone Dosing Using Wearable Device, the teachings of which are included herein in its entirety.

### TECHNICAL FIELD

The present subject matter relates to a wearable drug delivery system.

### BACKGROUND

Currently today there are only three ways to do Testosterone Replacement Therapy (TRT), namely, topical, an injectable with a synthetic grapeseed oil or cottonseed oil form, and Bio-Identical testosterone in the form of pellets. Bio-Identical testosterone is the safest form of testosterone.

### BRIEF DESCRIPTION OF THE DRAWINGS

The drawing figures depict one or more implementations, by way of example only, not by way of limitations. In the figures, like reference numerals refer to the same or similar elements.

Features of the various implementations disclosed will be readily understood from the following detailed description, in which reference is made to the appended drawing figures. A reference numeral is used with each element in the description and throughout the several views of the drawing. When a plurality of similar elements is present, a single reference numeral may be assigned to like elements, with an added letter referring to a specific element.

The various elements shown in the figures are not drawn to scale unless otherwise indicated. The dimensions of the various elements may be enlarged or reduced in the interest of clarity. The several figures depict one or more implementations and are presented by way of example only and should not be construed as limiting. Included in the drawing are the following figures:

FIG. 1A illustrates a drug delivery system configured to deliver a bio identical liquid hormone to a patient;

FIG. 1B illustrates a top view of the pump;

FIG. 1C illustrates a bottom view of the pump;

FIG. 1D illustrates a side view of the pump;

FIG. 1E illustrates a top view of the cartridge;

FIG. 1F illustrates a bottom view of the cartridge;

FIG. 1G illustrates a side view of cartridge; and

FIG. 2 is a method of delivering the bio identical liquid hormone to a patient.

### DETAILED DESCRIPTION

This disclosure includes a wearable TRT drug delivery system including a wearable pump configured to be worn by a user overnight. During the night the pump injects a prescribed per patient dosage of a liquid form bio-identical testosterone over a predetermined time period, such as a 6-hour period, mimicking the body's natural way of hormone development. This injection mimics the human process. The pump includes a cartridge pre-loaded with TRT from a pharmacy. Each cartridge has a 31-gauge needle approximately 3/16″-¼″ in length as well as an adhesive ring around the cartridge to ensure a proper seal to the body with a good attachment to keep from coming off in the night.

The following detailed description includes systems, methods, techniques, instruction sequences, and computing machine program products illustrative of examples set forth in the disclosure. Numerous details and examples are included for the purpose of providing a thorough understanding of the disclosed subject matter and its relevant teachings. Those skilled in the relevant art, however, may understand how to apply the relevant teachings without such details. Aspects of the disclosed subject matter are not limited to the specific devices, systems, and method described because the relevant teachings can be applied or practice in a variety of ways. The terminology and nomenclature used herein is for the purpose of describing particular aspects only and is not intended to be limiting. In general, well-known instruction instances, protocols, structures, and techniques are not necessarily shown in detail.

The terms “coupled” or “connected” as used herein refer to any logical, optical, physical, or electrical connection, including a link or the like by which the electrical or magnetic signals produced or supplied by one system element are imparted to another coupled or connected system element. Unless described otherwise, coupled or connected elements or devices are not necessarily directly connected to one another and may be separated by intermediate components, elements, or communication media, one or more of which may modify, manipulate, or carry the electrical signals. The term “on” means directly supported by an element or indirectly supported by the element through another element that is integrated into or supported by the element.

The term “proximal” is used to describe an item or part of an item that is situated near, adjacent, or next to an object or person; or that is closer relative to other parts of the item, which may be described as “distal.” For example, the end of an item nearest an object may be referred to as the proximal end, whereas the generally opposing end may be referred to as the distal end.

Additional objects, advantages and novel features of the examples will be set forth in part in the following description, and in part will become apparent to those skilled in the art upon examination of the following and the accompanying drawings or may be teamed by production or operation of the examples. The objects and advantages of the present subject matter may be realized and attained by means of the methodologies, instrumentalities and combinations particularly pointed out in the appended claims.

Reference now is made in detail to the examples illustrated in the accompanying drawings and discussed below.

In a person's prime testosterone is developed overnight while asleep making testosterone levels the highest of the day every morning.

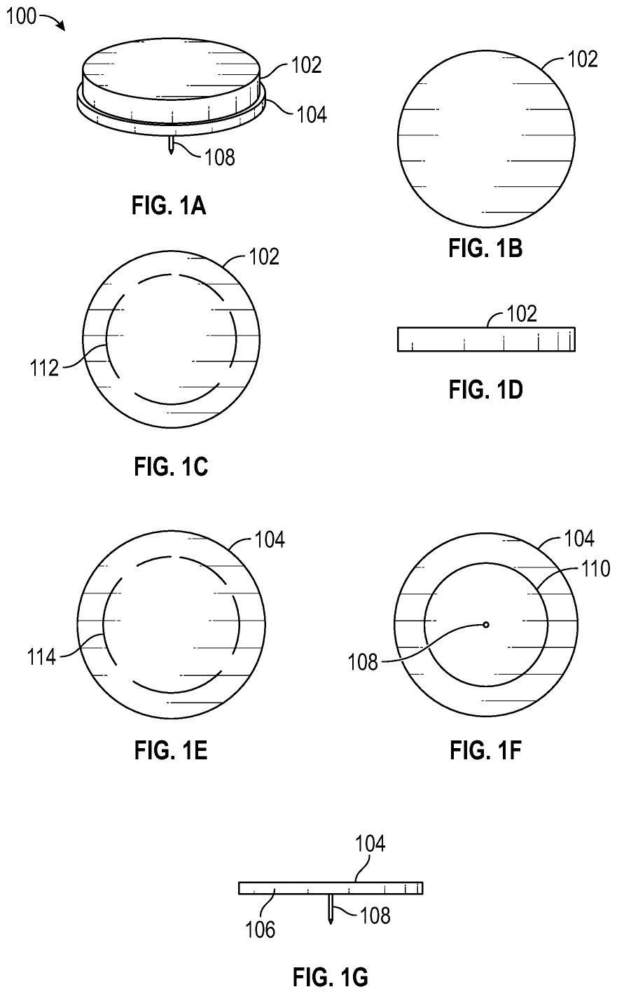

FIG. 1A illustrates a wearable TRT drug delivery system 100 including a wearable battery-operated pump 102, also referred to as an injector, configured to be worn by a user overnight, and a cartridge 104. The battery (not shown) is rechargeable and replaceable. During the night pump 102 injects a prescribed per patient dosage of a bio-identical liquid hormone 106 stored in cartridge 104, such as testosterone over a predetermined time period, such as a 6-hour period, mimicking the body's natural way of hormone development. The 6-hour period is critical because it matches the body's generation rate of testosterone overnight. This injection process mimics the process of the human body. Pump 102 includes cartridge 104 pre-loaded with a bio-identical liquid form of human TRT 106, such as available from Sexual Wellness Centers of America, Frisco, Texas, the Applicant of this application. The bio-identical liquid hormone 106 is a saline cholesterol-based form of testosterone and estrogen (if needed for women). The bio-identical liquid hormone 106 includes a saline solution to create the liquid, and the cholesterol helps the body absorb the liquid bio-identical hormone. Liquid hormone 106 is available from

Each cartridge 104 has a 31-gauge needle 108 approximately ¼″- 3/16″ in length as well as an adhesive ring 110 around cartridge 104 to ensure a proper seal of needle 108 to the user's body with a good attachment to keep cartridge 104 of drug delivery system 100 coming off the body in the night. The bio-identical liquid hormone 106 allows the significantly small 31-gauge needle 108 to inject the hormone to a user's body. An attachment member 112 on the bottom of pump 102 is configured to selectively connect to a mating attachment member 114 on the top of cartridge 104 to pump 102 such that bio-identical hormone 106 is fluidly connected to pump 102 and configured to be dispensed by pump 102 via needle 108.

To make delivery system 100 duplicate the body's natural development of testosterone, delivery system 100 is the first bio-identical form of injectable liquid testosterone fine enough to be administered through a very small 31-gauge needle 108. Currently, the only injectable testosterone available is not only synthetic, but it requires a large 22-gauge needle to load the syringe and a maximum of 27-gauge to inject into the body. The common form of testosterone is very thick gel due to the oil base it is made from, and thus larger needles are required for delivery. The bio-identical liquid hormone 106 is critical to allow the significantly small 31-gauge needle 108 and pump 102 to be used to inject the hormone to the body. The 31-gauge needle 108 is a critical size to reduce pain from the needle to the user, and to be comfortably used in a small attachable pump.

This bio-identical testosterone delivery process includes a user wearing the delivery system 100 5-6 nights per week depending on severity of testosterone deficiency.

Pump 102 is the preferably the size of three silver dollars stacked up. The user attaches a cartridge 104 having a preloaded predetermined dose of bio-identical liquid testosterone 106 of their personalized daily dose to pump 102 and sticks the 31-gauge needle 108 anywhere there is a layer of fat tissue, e.g., the tricep, the buttocks, and the stomach, preferably anywhere it would be comfortable to the user during sleep. Pump 102 and needle 108 are structured to deliver the bio-identical liquid testosterone 106 at a rate such that the liquid predetermined dose of bio-identical testosterone 106, such as 2 cc, is delivered in a predetermined time period, such as 6-hours.

FIG. 1B illustrates a top view of pump 102.

FIG. 1C illustrates a bottom view of pump 102.

FIG. 1D illustrates a side view of pump 102.

FIG. 1E illustrates a top view of cartridge 104.

FIG. 1F illustrates a bottom view of cartridge 104.

FIG. 1G illustrates a side view of cartridge 104.

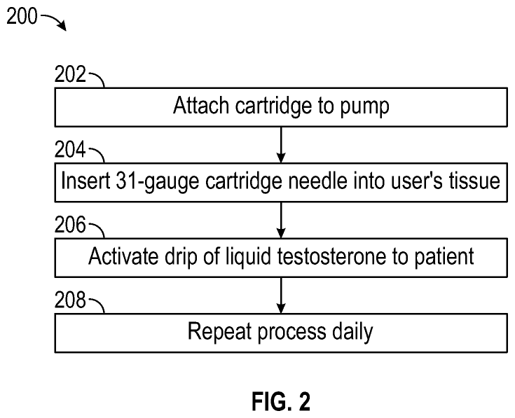

FIG. 2 illustrates method 200 of using drug delivery system 100.

At step 202, cartridge 104 containing hormone 106 is selectively attached by the user to pump 102. The attachment member 112 of pump 102 is attached to attachment member 114 of cartridge 104 such that the pump 102 is fluidly coupled to bio-identical hormone 106 and configured to dispense bio-identical hormone 106 via needle 108.

At step 204, the user inserts a 3/16″ long 31-gauge needle 108 into the skin of the user and secures cartridge 104 to the user using adhesive strip 110.

At step 206, once attached to the body, the user activates pump 102, such as pressing a button, to start a 6-hour drip of the liquid bio-identical hormone 106 into the user via needle 108. The 6-hour drip restores the user's testosterone level and mimics the body's natural development of testosterone. Delivery system 100 is the first bio-identical form of delivering testosterone to the body.

At step 208, in the morning the user removes and discards the cartridge 104 and repeats the method 200 the next night before bed.

The bio-identical liquid hormone 106 is a saline cholesterol-based form of testosterone and estrogen (if needed for women) as described. This bio-identical liquid hormone 106 is critical to allow the use of the 31-gauge cartridge needle 108. This disclosure is the first and only bio identical way of dispensing hormones with a bio identical form of hormones. This process mimics the body's way of distributing hormones similar to when they were in their prime, while they sleep and over the same approximate time frame.

This delivery system 100 and process is beneficial because hormone levels will not drop from optimal levels save what they use during the day and will be replenished as they sleep.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than al features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. A wearable drug delivery system, comprising:
a pump;
a cartridge selectively coupled to the pump, the cartridge holding a liquid form of a bio-identical human hormone; and
a needle coupled to the cartridge and configured to deliver the liquid form of the bio-identical human hormone to a user.

2. The wearable drug delivery system of claim 1, wherein the liquid form of bio-identical hormone is a cholesterol-based form of testosterone.

3. The wearable drug delivery system of claim 1, wherein the liquid form of bio-identical hormone is a cholesterol-based form of testosterone and estrogen.

4. The wearable drug delivery system of claim 1, wherein the needle is a 31-gauge needle.

5. The wearable drug delivery system of claim 2, wherein the needle is a 31-gauge needle.

6. The wearable drug delivery system of claim 1, wherein the pump is configured to be worn on the user.

7. The wearable drug delivery system of claim 1, wherein the pump and the needle are configured to deliver the liquid form of the bio-identical human hormone over a predetermined time period.

8. The wearable drug delivery system of claim 7, wherein the predetermined time period is 6-hours.

9. A method of delivering a liquid form of a bio-identical hormone to a user using a wearable drug delivery system comprising a pump, a cartridge selectively coupled to the pump, the cartridge holding a liquid form of a bio-identical human hormone, and a needle coupled to the cartridge and configured to deliver the liquid form of the bio-identical human hormone to a user, the method comprising;
attaching the drug delivery system to a user; and
delivering the liquid form of the bio-identical human hormone to a user.

10. The method of delivering of claim 9, wherein the liquid form of bio-identical hormone is a cholesterol-based form of testosterone.

11. The method of claim 9, wherein the liquid form of bio-identical hormone is a cholesterol-based form of testosterone and estrogen.

12. The method of claim 9, wherein the needle is a 31-gauge needle.

13. The method of claim 10, wherein the needle is a 31-gauge needle.

14. The method of claim 9, wherein the pump is configured to be worn on the user.

15. The method of claim 9, wherein the pump and the needle are configured to deliver the liquid form of the bio-identical human hormone over a predetermined time period.

16. The method of claim 15, wherein the predetermined time period is 6-hours.
